(19) United States
(12) Patent Application Publication
(10) Pub. No.: US 2015/0313949 A1
(43) Pub. Date: Nov. 5, 2015

(54) Method for Metabolic Correction

(71) Applicant: Infinity Health Advisors, LLC, Lancaster, PA (US)

(72) Inventor: Dawn Marie Cutillo, Ephrata, PA (US)

(73) Assignee: INFINITY HEALTH ADVISORS, LLC, Lancaster, PA (US)

(21) Appl. No.: 14/700,816

(22) Filed: Apr. 30, 2015

Related U.S. Application Data
(60) Provisional application No. 61/987,870, filed on May 2, 2014.

(51) Int. Cl. A61K 35/55; A61K 35/22; A23L 1/29; A61K 31/57

(52) U.S. Cl. CPC A61K 35/55; A61K 31/57; A61K 35/22; A23L 1/293; A23V 2002/00

(57) ABSTRACT

A method of weight loss is disclosed having a weight loss phase including the steps of taking at least one glandular supplement daily, and eating a ketogenic diet.

### CROSS-REFERENCE TO RELATED APPLICATIONS

This application claims the benefit of the filing date under 35 U.S.C. §119(a)-(d) of U.S. Patent Application No. 61/987,870, filed May 2, 2015.

### FIELD OF THE INVENTION

The invention is generally related to a method for metabolic correction, and, more specifically, to a method of weight loss through metabolic correction.

### BACKGROUND

There is a growing epidemic of women entering the peri-menopausal and menopausal years who are unable to effectively lose weight through traditional weight loss methods, such as low calorie diets, ketogenic diets, or vigorous cardiovascular exercise. These traditional weight loss methods are often not effective in peri-menopausal and menopausal women, because they fail to address underlying hormonal imbalances responsible for weight retention.

Cortisol, estrogen, and progesterone are three critical hormones in females. Normally these hormones are in equilibrium with each other. However, poor diet and high stress disrupt the normal balance of these hormones, causing one's energy level and metabolism rate to drop dramatically. The result is that the traditional weight loss methods are not effective.

Cortisol is a stress induced hormone produced in the adrenal glands. In the presence of stress, the hypothalamus gland secretes a corticotrophin-releasing hormone, which stimulates the adrenal glands to make cortisol. Cortisol initiates glycogenolysis, which is the rapid production of glucose from glycogen, by activating glycogen phosphorylase. Glycogen phosphorylase works in combination with the neurotransmitter epinephrine (adrenaline) to facilitate the rapid production of glucose. This rapid production is colloquially described as an “adrenalin rush”, which is the effect felt by a person when the body is rapidly producing glucose to respond to the applied stress.

Additionally, cortisol counteracts insulin. Insulin is secreted by the pancreas in response to high levels of blood sugar. Insulin works to reduce blood sugar levels by activating the fat storage processes in fat cells. Excess blood sugar is converted into fatty acids and stored as triglycerides. When blood sugar drops, cortisol is secreted by the adrenal glands to raise blood sugar levels by metabolizing glycogen into glucose.

Estrogen and progesterone are female sex hormones that counteract each other during different phases of the menstrual cycle. Estrogen stimulates growth of the uterine lining (endometrium), causing it to thicken during the pre-ovulatory phase of the menstrual cycle. Additionally, estrogen is a factor in inducing physiological changes in the breast, vagina, skin, bone, blood vessels, and other tissues in preparation for pregnancy.

Progesterone stimulates the uterine lining to secrete special proteins during the second half of the menstrual cycle, to prepare for the implantation of a fertilized egg. Beyond preparation for pregnancy, progesterone has a multitude of effects throughout the body, many of which are attributed to counteracting the physiological changes induced by estrogen.

Consequently, if the level of progesterone drops while the level of estrogen remains constant, “estrogen dominance” occurs, where the balance of estrogen to progesterone has shifted in favor of estrogen. Since estrogen is a factor in inducing a variety of physiological changes throughout the body, without sufficient progesterone levels to counter these effects, an estrogen dominant female will often experience a variety of undesirable effects. These can include an increase in peri-menopausal and menopausal symptoms, decreased libido, fatigue, headaches, and irritability. In addition, for women trying to lose weight, this estrogen dominance can be even more frustrating, because excess estrogen is known to stimulate insulin production in the pancreas. The insulin prevents estrogen dominant women from undergoing ketosis, by instead promoting the conversion of sugars into fat, effectively turning off the body's ability to efficiently use fat as an energy source. The effect is that the estrogen dominate female will often see fat gains, even when engaging in traditional weight loss methods.

Modern lifestyles are characterized by high stress environments and diets high in refined foods. The high stress causes the hypothalamus gland to constantly stimulate the adrenal glands to produce cortisol. The frequent consumption of refined foods spike blood sugar levels, causing a surge in insulin production, which lowers blood sugar levels by converting the excess sugar into body fat. The blood sugar levels then drop to low levels, which stimulates the production of cortisol to raise the blood sugar levels. For individuals in high stress environments who have diets high in refined foods, the constant rise and fall activity of blood sugar levels will cause the adrenal glands to constantly produce cortisol, eventually stressing the adrenal glands and leading to adrenal fatigue. Once adrenal fatigue occurs, when stress is applied, the body is unable to make enough cortisol to deal with the stress, and the person responds poorly to and “crashes” during the stress.

Adrenal fatigue has additional, undesirable consequences for females. Since the adrenal gland is unable to produce enough cortisol to meet the hyphothalamus gland's demand, the adrenal gland short circuits the normal synthetic pathway used to make cortisol. Progesterone is a late stage, synthetic precursor of cortisol, so the fatigued adrenal glands will consume the body's available progesterone to make cortisol more quickly. While this allows the adrenal glands to use a quicker synthetic route to cortisol, the balance of estrogen to progesterone is shifted towards estrogen, resulting in the manifestation of estrogen dominance.

Hormone Replacement Therapy (HRT) is a commonly prescribed method of addressing estrogen and progesterone imbalances, by simply taking an estrogen or progesterone supplement to raise the levels in the body. While this approach may relieve hot flashes, mood swings, and other menopausal symptoms, the approach fails to address the root causes of the imbalance. Alarmingly, HRT has been linked to a rise in heart disease and breast cancer. For females engaged in weight loss programs, HRT has also been shown to increase insulin levels.

Traditional weight loss methods such as low calorie diets, ketogenic diets, or vigorous exercise are ineffective in women suffering from estrogen dominance, because these women are unable to undergo ketosis. While low calorie and ketogenic diets may slow the weight gain experienced by estrogen dominant women, neither is effective in promoting weight loss, because high insulin levels prevent the body from entering into ketosis.

Vigorous exercise also fails to promote weight loss for the same reason, and can actually have deleterious effects. Vigorous exercise is treated as an applied stress on the body, which stimulates cortisol production. In females suffering from estrogen dominance and/or adrenal fatigue, further cortisol production will exacerbate the effects of estrogen dominance, as the fatigued adrenal glands reduce progesterone levels by producing cortisol.

There is a need in the art for a method of weight loss in per-menopausal and menopausal women that resets the hypothalamus gland's stimulation of cortisol product, lowers the level of stress hormone production like cortisol, lowers the level of insulin production, restores the estrogen and progesterone balance, and promotes ketosis.

### SUMMARY

A method of weight loss has a weight loss phase including the steps of taking at least one glandular supplement daily, and eating a ketogenic diet.

### DETAILED DESCRIPTION

A method of weight loss in peri-menopausal and menopausal females is provided. The method includes a weight loss phase, a maintenance phase, and a lifestyle phase.

Exemplary steps in the weight loss phase include taking at least one glandular supplement, eating a ketogenic diet, performing a conditioned response relaxation program, and engaging in minimal cardiovascular physical activity. In one embodiment, the weight loss phase is approximately 27-31 days in length. In another embodiment, the weight loss phase is approximately 28-30 days in length. One of ordinary skill in the art would understand that the length of the weight loss phase will depend on the amount of weight an individual needs to lose, with shorter lengths of time for individuals with less weight to lose, and longer lengths of time for individuals with more weight to lose.

In an exemplary embodiment, the glandular supplement may include hypothalamus, pituitary, thyroid, or adrenal glandulars, or any combination thereof. The glandulars are made from the organs and tissues of mammals, such as bovine, porcine, or ovine sources, or any combination thereof. While the exact physiological mechanism is unclear, glandulars are known to strengthen and support the function of that tissue or organ in a human. The purpose of the glandulars supplement is to strengthen and support an overactive or fatigued hypothalamus, pituitary, thyroid, or adrenal gland. By strengthening and supporting these glands, the glands are reset and their normal function is restored. By supporting the glands, rather than supplementing deficient hormones directly through HRT, the body is able to restore natural glandular function. In an embodiment, the concentration of glandular supplements is 1×-10× for each included glandular, such as the hypothalamus, pituitary, thyroid, and adrenal glandulars. In another embodiment, the concentration of glandular supplement is 3×-8× for each included glandular. In another embodiment, the concentration of the glandular supplement is 4×-8× for each included glandular. In yet another embodiment, the concentration of the glandular supplement is 6× for each included glandular. The glandular supplements are dosed one, two, three, or four times daily during the weight loss phase. In an embodiment, the dose is 5-15 drops. In another embodiment, the dose is 7-10 drops. In an embodiment, the glandular supplement includes further includes progesterone or a progesterone precursor in combination with the included glandular or combination of glandulars.

The ketogenic diet is a diet that is designed to induce the body into a metabolic state of ketosis. In ketosis, the body is primarily using fat for energy production. The source of the fat can be from external food sources, or from fat reserves stored in the body. When the body uses the fat reserves stored in the body, weight loss occurs in that individual.

In an exemplary embodiment of the ketogenic diet, a four (4) to one (1) ratio of fat to carbohydrate and protein (combined) is followed. For example, an individual requiring a two thousand (2,000) calories daily, two hundred and fifty (250) calories come from carbohydrates, two hundred and fifty (250) calories come from protein, and fifteen hundred (1500) calories from fat. In the exemplary ketogenic diet, only the carbohydrates and protein are consumed from external sources, whereas the individual's body fat supplies the 1500 calories from fat. In an exemplary 2000 calorie ketogenic diet, an individual will consume daily, approximately seven (7) ounces of a protein sources, such as meat, fish, or poultry. The individual will also consume daily, approximately three (3) cups of raw or cooked vegetables, one (1) stemmed or peeled fruit, one (1) cup of lose fruit, such as berries, and a starch, such as a rice or gluten-free cracker (starch) carbohydrate sources. The protein and carbohydrates can be consumed throughout the day, or into meals of a portion of the protein, one fruit, one vegetable, and a partial starch.

In another exemplary embodiment, the ketogenic diet includes approximately 200-300 calories from carbohydrates and approximately 200-300 calories from proteins, with the balance of fat being from the individual's body fat. In an embodiment, the ketogenic diet includes approximately 200-600 calories from carbohydrates and approximately 200-600 calories from proteins, with the balance of fat being from the individual's body fat. In another exemplary embodiment, the ketogenic diet includes approximately 250 calories from carbohydrates, and approximately 250 calories from proteins, with the balance of fat being from the individual's body fat.

The conditioned response relaxation program includes sound-wave therapy to psychologically calm the mind and reduce stress. In another embodiment, the conditioned response relaxation program further includes aromatherapy to physiologically relax the body and reduce stress.

The sound-wave therapy employs the concept brainwave entrainment that an auditory stimulus having a frequency within the frequency range of brainwaves, will cause the predominant brainwave frequency to move towards the frequency of the auditory stimulus. This process is known as brainwave entrainment. Since the human ear is limited to perceiving frequencies from approximately 20 Hz to 20,000 Hz, and the frequency of the human brain waves are generally below 40 Hz, binaural beats are used.

Under brainwave entrainment, a first audio signal at a first frequency is applied to one ear of an individual. A second audio signal close in frequency to the first audio signal, is applied to the second ear of the individual at the same time as the first frequency is applied to the first ear. The brain combines the two frequencies to produce a third frequency pulse, called a binaural beat. The binaural beat is the difference between the first and second frequencies. For example, if the first frequency applied is 505 Hz, and the second frequency is 515 Hz, the binaural beat frequency is the difference of the first and second frequency, which would be 10 Hz.

Research has shown that the brainwave frequency changes depending on the activity of the individual. For example, when a person is relaxing while awake, about to fall asleep, about to wake up, or in rapid eye motion sleep (REM), the brainwaves have a frequency range of approximately 7-13 Hz, known as an Alpha frequency. When a person is in a meditative state or in non-REM sleep, the brainwaves have a frequency range of approximately 4-7 Hz, known as a Theta frequency. When a person is in a deep dreamless sleep, the brainwaves have a frequency of <4 Hz, known as a Delta frequency.

The sound wave therapy step uses brain entrainment to produce a binaural beat having a frequency of approximately the 4-13 Hz (the Alpha and Theta frequency ranges), to cause the predominant brainwave frequency to move towards the Alpha and Theta frequency ranges. By shifting an individual's predominant brainwave frequency into the Alpha and Theta frequency ranges, the individual will relax, which will encourage the body to recover from applied stresses.

The conditioned response relaxation program step is performed during time when stress levels, and consequently high cortisol levels, are typically the highest during the day. In an embodiment, the conditioned response relaxation program step is performed in the afternoon, such as between 2-4 pm, or 3 pm. In another embodiment, the conditioned response relaxation program step is performed in the evening before sleeping. In yet another embodiment, the conditioned response relaxation program step is performed in both the afternoon and in the evening before sleeping.

In an embodiment of the conditioned response relaxation program step, aromatherapy is used in concert with the sound wave therapy. Aromatherapy oils, also known as essential oils, are known to physiologically relax the body, and help assist individuals in relieving stress. Using the aromatherapy oils in concert with the sound wave therapy helps the individual relax and relieve stress. Further, after a period of time, the individual will develop a Pavlovian conditioned response to the scent of the aromatherapy oils, and use of the aromatherapy oil alone will bring them back into a state of relaxation.

During the weight loss phase, physical activity is minimized, by limiting the exercise to minimal movement methods, such as yoga, Pilates, rebounding, or walking Vigorous cardiovascular physical activity is minimized because it is an applied stress on the body, causing cortisol levels to rise and blood sugar levels to subsequently increase, resulting in an increase in insulin production. Since insulin production prevents the body from entering into ketosis, vigorous cardiovascular physical activity needs to be minimized to promote weight loss.

Under the weight loss phase of the method, the stress level of an individual are reduced through methods such as the conditioned response relaxation program step and/or the minimal vigorous cardiovascular physical activity. By removing the stresses, the hypothalamus gland will reduce the adrenal glands' cortisol production levels, allowing the hypothalamus gland to rest and the adrenal glands to recover from the adrenal fatigue. The glandulars supplement strengthens and supports the overactive or fatigued hypothalamus, pituitary, thyroid, and adrenal glands. By strengthening and supporting these glands, the glands are able to reset and restore their normal function.

Consequently, by lowering cortisol production levels, progesterone levels will increase to a normal level that balances against estrogen, because the adrenal glands are not robbing progesterone levels to produce cortisol. Estrogen dominance decreases, along with many of the undesirable physical affects experience by women having estrogen dominance. Further, by lowering cortisol levels, spikes in insulin levels are also reduced, turning off the body's fat storage mechanisms, and allowing the individual to maintain ketosis and lose weight.

The ketogenic diet further enhances weight loss, because refined foods are excluded from the individual's diet. Blood sugar levels therefore remain constant and recurring insulin level spikes are reduced, allowing the individual to maintain ketosis and lose weight. Further, the body is able to maintain ketosis, the individuals often burn 1500+ calories derived directly from body fat.

Upon completion of the weight loss phase, the maintenance phase begins. Exemplary steps in the maintenance phase include introducing dairy and nuts into the ketogenic diet, performing a food sensitivity test on each dairy and nut introduced into the ketogenic diet, testing for Candida yeast, taking a digestive probiotic, discontinuing the daily taking of the glandular supplement, applying a topical cortisol building block crème; and applying a topical phyto-estrogen and progesterone crème. In one embodiment, the maintenance phase further includes the step of continuing the conditioned response relaxation program step.

In one embodiment, the maintenance phase is approximately 19-23 days in length. In another embodiment, the maintenance phase is approximately 21 days in length.

During the maintenance phase, dairy and nut products are introduced into the ketogenic diet, and the food sensitivity test is performed on each dairy and nut introduced. The term “food sensitivity” refers to foods that elicit an immune response, as well as gas, bloating, headaches, or general nervous system disruptions. Such foods serve as an applied stress on the body, causing cortisol production levels to increase. Food products that cause food sensitivity symptoms are removed from the diet, to prevent excess cortisol production from effecting the balance of estrogen to progesterone.

An embodiment of the food sensitivity test is a pulse test, such as a Coca Pulse test. The Coca Pulse test is premised on the idea that stress caused by one's nervous system in response to a food product will increase one's resting pulse rate. Sensitivity to a food product is determined when a food product increases the resting pulse rate by more than 6 beats per minute. Such food products should be eliminated from one's diet.

The Coca Pulse test is performed by first taking and recording one's pulse fourteen (14) times per day, for three consecutive days: once in the morning upon waking and before getting out of bed, once before each meal, three (3) times after each meal at thirty (30) minute intervals, and one just prior before going to bed. The pulse is measured for an entire minute, during which one is in a seated or reclined and relaxed position. The foods consumed during each meal are recorded. This process is performed for two (2) to four (4) days. Any meals that exhibit a pulse rate increase of more than six (6) beats per minute indicates the presence of a suspected food sensitivity. After the 2-4 days, meals that exhibited a pulse rate increase of more than 6 beats per minute are compared to determine any common food products. Suspected food products are then pulse tested individually.

To test a food product individually, one consumes the suspected food product once each day for several days. One's pulse is taken just prior to consuming the food product, and taken 3 times at 30 minutes intervals. Any foods that are consistently accompanied by an elevated pulse of 6 beats per minute or greater, may indicate a food sensitivity, and should be avoided.

During the maintenance phase, testing for Candida yeast is performed. While yeast is naturally present in the digestive system, the presence of high levels of Candida yeast will cause the body to retain fluid, and will increase an individual's sugar cravings, because sugars are being consumed by the Candida yeast, rather than being digested. During the weight loss phase, Candida levels will decrease, due to the lack of sugars being consumed. When the Candida yeast testing shows higher than baseline levels, the individual takes a digestive probiotic to help restore a natural balance of digestive flora. In one embodiment, the digestive probiotic includes Lactobacillus acidophilus.

During the maintenance phase, the glandular supplement is discontinued as the hypothalamus and adrenal glands normal function is restored. In an embodiment of the maintenance phase, a topical cortisol building block crème is applied to provide the adrenal glands with the building blocks necessary to produce cortisol, so that progesterone reserves are not used. In an embodiment, the topical cortisol building block crème includes pregnenolone in an therapeutically effective dose. In an embodiment, the dose of the cortisol building block crème includes 1-10 ng of pregnenolone. In another embodiment, the dose of the cortisol building block crème includes 5 ng of pregnenolone. The dose is taken 1, 2, 3, or 4 times per day. In one embodiment, the cortisol building block crème further includes maca extract.

In another embodiment of the maintenance phase, a topical phyto-estrogen and progesterone crème is used to relieve menstruating individuals suffering from the effects of PMS symptoms and/or endometriosis. The progesterone assists the body in maintaining the balance of estrogen to progesterone. The amount of progesterone in a dose of the crème is a therapeutically effective amount. In one embodiment, the dose of the crème includes 5-30 mg of progesterone. In another embodiment, the dose of the crème includes 10-20 mg of progesterone. In yet another embodiment, the dose of the crème includes 10 mg of progesterone. In another embodiment, the dose of the crème includes 20 mg of progesterone. The dose is taken 1, 2, 3, and 4 times per day.

In an embodiment, the phyto-estrogen is 7-oxo-dehydroepiandrosterone (DHEA), which binds to estrogen receptors, causing the body to reduce estrogen levels. The amount of DHEA in a dose of the crème is a therapeutically effective amount. In one embodiment, the dose of DHEA is 10-20 mg. In another embodiment, the dose of DHEA is 15 mg. The dose is taken 1, 2, 3, or 4 times per day.

During the maintenance phase, similar to the weight loss phase, vigorous cardiovascular physical activity is minimized to avoid adding applied stress on the body.

Upon completion of the maintenance phase, the lifestyle phase begins. Exemplary steps in the lifestyle phase include introducing vegetable starches, non-wheat gluten starches, and wheat products into the ketogenic diet, performing the food sensitivity test on each vegetable starch, non-wheat gluten starch, and wheat product introduced into the ketogenic diet, testing for Candida yeast, and taking a digestive probiotic. In an embodiment, the lifestyle phase further includes the step of continuing the application of the topical cortisol building block crème, as described in the maintenance phase. In another embodiment, the lifestyle phase further includes the step of continuing the application of the topical phyto-estrogen and progesterone crème, as described in the maintenance phase. In yet another embodiment, the lifestyle phase further includes the step of continuing the conditioned response relaxation program step, as described in the maintenance phase. In another embodiment, the lifestyle phase further includes the step of continuing minimal cardiovascular physical activity to continue to avoid adding applied stress on the body.

In an embodiment, the duration of the lifestyle phase is ongoing.

In an embodiment, the vegetable starches, non-wheat gluten starches, and wheat products are introduced into the individual's diet over one week. In another embodiment the starches and wheat products are introduced into the individual's diet over two weeks. In yet another embodiment, the starches and wheat products are introduced into the individual's diet over three weeks. In yet another embodiment, the starches and wheat products are introduced into the individual's diet over four or more weeks.

The food sensitivity test used during the lifestyle phase is substantially the same as the Coca Pulse test described in the maintenance phase.

The testing for Candida yeast and taking of a digestive probiotic during the lifestyle phase is substantially the same as described in the maintenance phase.

In an embodiment of the lifestyle phase, cardiovascular physical activity continues to minimized. However, in an embodiment, the lifestyle phase further includes the step of resistance training

As detailed above, traditional weight loss methods such as low calorie diets, ketogenic diets, or vigorous exercise are ineffective in women suffering from estrogen dominance, because these methods fail to address the root causes of weight retention in peri-menopausal and menopausal females. The disclosed method of weight loss provides advantages over the traditional weight loss methods, because the combination of steps addresses the hormonal imbalance causing weight retention in peri-menopausal and menopausal women.

The method further provides advantages of traditional hormone balancing therapies such as Hormone Replacement Therapy, which involves injecting deficient hormones, without addressing the root causes of the hormone imbalance. The method eliminates the artificial hormone supplements and dangerous side effects of HRT by allowing the individual's own body to stabilize and balance the hormones naturally. The method resets the hypothalamus gland's stimulation of cortisol product, lowers the level of stress hormone production like cortisol, lowers the level of insulin production, restores the estrogen and progesterone balance, and promotes ketosis.

While the invention has been described in detail and with reference to specific embodiments, one of ordinary skill in the art would appreciated that the described embodiments are illustrative, and that various changes and modifications can be made without departing from the scope of the invention.

For example, while the above method has been described in the context of a peri-menopausal and menopausal women, the method is not limited to this demographic. The method can also be applied to weight loss in males as well. In a male, all of the above steps are followed, except during the maintenance phase. The use of the topical phyto-estrogen and progesterone crèmes is omitted for males during the maintenance phase.

What is claimed is:

1. A method of weight loss, comprising the step of:
applying a weight loss phase including
taking at least one glandular supplement daily, and
eating a ketogenic diet.

2. The method of weight loss of claim 1, wherein the glandular supplement includes hypothalamus, pituitary, thyroid, or adrenal glandulars, or a combination thereof.

3. The method of weight loss of claim 2, wherein the glandular supplement further includes progesterone in combination with the glandulars.

4. The method of weight loss of claim 1, wherein the ketogenic diet includes approximately a four to one ratio of fat to carbohydrate and protein.

5. The method of weight loss of claim 4, wherein the ketogenic diet includes approximately 200-300 calories from carbohydrates and approximately 200-300 calories from proteins.

6. The method of weight loss of claim 5, wherein the ketogenic diet includes approximately 250 calories from carbohydrates and approximately 250 calories from proteins.

7. The method of weight loss of claim 1, wherein the weight loss phase further includes the step of performing a conditioned response relaxation program.

8. The method of weight loss of claim 7, wherein the conditioned response relaxation program includes sound-wave therapy.

9. The method of weight loss of claim 8, wherein the conditioned response relaxation program is performed in the afternoon, evening, or both in the afternoon and the evening.

10. The method of weight loss of claim 1, wherein the weight loss phase is approximately 27-31 days in length.

11. The method of weight loss of claim 1, further comprising the steps of:
applying a maintenance phase after the weight loss phase including
introducing dairy and nuts into the ketogenic diet, and
performing a food sensitivity test on each dairy and nut introduced into the ketogenic diet.

12. The method of weight loss of claim 11, wherein the maintenance phase further includes:
testing for Candida yeast; or
taking a digestive probiotic; or a combination thereof.

13. The method of weight loss of claim 11, wherein the maintenance phase further includes:
discontinuing the daily taking of the at least one glandular supplement;
applying a topical cortisol building block crème; or
applying a topical phyto-estrogen and progesterone crème; or a combination thereof.

14. The method of weight loss of claim 11, wherein the maintenance phase is approximately 21 days in length.

15. The method of weight loss of claim 11, further comprising the step of
applying a lifestyle phase after the weight loss phase including
introducing vegetable starches, non-wheat gluten starches, and wheat products into the ketogenic diet; and
performing the food sensitivity test on each vegetable starch, non-wheat gluten starch, and wheat product introduced into the ketogenic diet.

16. The method of weight loss of claim 15, wherein the lifestyle phase further includes:
testing for Candida yeast, or
taking a digestive probiotic, or a combination thereof.

17. The method of weight loss of claim 1, further comprising the step of engaging in physical activity.

18. A method of weight loss, comprising the steps of:
applying a weight loss phase including
taking at least one glandular supplement daily, and
eating a ketogenic diet;
applying a maintenance phase including
introducing dairy and nuts into the ketogenic diet,
performing a food sensitivity test when each dairy and nut are introduced into the ketogenic diet,
testing for Candida yeast, and
taking a digestive probiotic;
applying a lifestyle phase including
introducing vegetable starches, non-wheat gluten starches, and wheat products into the ketogenic diet;
performing the food sensitivity test on each vegetable starch, non-wheat gluten starch, and wheat product introduced into the ketogenic diet,
testing for Candida yeast, and
taking a digestive probiotic.

19. The method of weight loss of claim 18, further comprising the step of engaging in physical activity.
